(19) United States
(12) Patent Application Publication
(10) Pub. No.: US 2021/0290144 A1
(43) Pub. Date: Sep. 23, 2021

(54) MONITORING DEVICE AND METHOD FOR CONTROLLING THRESHOLD THEREOF

(71) Applicant: JIANGSU BAINING YINGCHUANG MEDICAL TECHNOLOGY CO., LTD., Nanjing (CN)

(72) Inventors: Zhigang SHI, Nanjing (CN); Jiancong LI, Nanjing (CN)

(73) Assignee: JIANGSU BAINING YINGCHUANG MEDICAL TECHNOLOGY CO., LTD., Nanjing (CN)

(21) Appl. No.: 17/342,712

(22) Filed: Jun. 9, 2021

Related U.S. Application Data
(63) Continuation of application No. PCT/CN2019/115537, filed on Nov. 5, 2019.

(30) Foreign Application Priority Data
Dec. 26, 2018 (CN) 201811600448.1
May 9, 2019 (CN) PCT/CN2019/086104

(51) Int. Cl. A61B 5/395; A61B 5/00

(52) U.S. Cl. CPC A61B 5/395; A61B 5/0002; A61B 2562/227; A61B 5/7475; A61B 5/746

(57) ABSTRACT

The present disclosure discloses a monitoring device and a method for controlling a threshold thereof. The monitoring device may include a host. The monitoring device may also include an information acquisition module connected to the host via an electrical signal. The information acquisition module may be configured to acquire an electromyographic signal. The host may include a signal processing module configured to process the electromyographic signal to determine monitoring information corresponding to the electromyographic signal. The monitoring device may further include an output module connected to the signal processing module via an electrical signal. The output module may at least be configured to output the monitoring information. The method may include setting a threshold of the monitoring device, placing an electrode to the target area, and starting the monitoring device. The monitoring device may output prompt information based on the threshold.

### CROSS-REFERENCE TO RELATED APPLICATION

This application is a continuation of International Patent Application No. PCT/CN2019/115537, filed on Nov. 5, 2019, which claims priority of Chinese Patent Application No. 201811600448.1, filed on Dec. 26, 2018, and International Patent Application No. PCT/CN2019/086104, filed on May 9, 2019, the contents of each of which are hereby incorporated by reference.

### TECHNICAL FIELD

The present disclosure generally relates to medical technology, and in particular, to a monitoring device and a method for controlling a threshold thereof.

### BACKGROUND

During surgery, nerves of patients may be damaged for a variety of reasons. For example, a surgeon may pull a nerve when the surgeon is unclear about a location of the nerve, causing damage to the nerve. As another example, cold/hot water to clean tissue may stimulate relevant nerves. If no one finds there is an external stimulation on a nerve for a long time, it may cause damage to the nerve.

Therefore, it is desirable to provide a solution, by which nerves can be identified and positioned, and/or whether a nerve is stimulated by an external stimulation can be monitored, thereby protecting the nerve of a patient in surgery.

### SUMMARY

Embodiments of the present disclosure provides a monitoring device. The monitoring device may include a host; and an information acquisition module connected to the host via an electrical signal, the information acquisition module being configured to acquire an electromyographic signal from a target area. The host may include a signal processing module configured to process the electromyographic signal to determine monitoring information corresponding to the electromyographic signal. The monitoring device may further include an output module connected to the signal processing module via an electrical signal. The output module at least be configured to output the monitoring information.

In some embodiments, the information acquisition module may include an electrode. The electrode may be configured to acquire an electromyographic signal generated by an external stimulation and transmit the electromyographic signal to the signal processing module.

In some embodiments, a value of the electromyographic signal acquired by the electrode may range from 5 μV to 1 mV.

In some embodiments, the electrode may transmit the electromyographic signal to the signal processing module in a wire or wireless manner.

In some embodiments, a material of the electrode may include medical stainless steel and high conductive rubber.

In some embodiments, a connection interface may be provided on the host. The connection interface may be configured to make the electrode in a direct connection with a main board of the host.

In some embodiments, the direct connection may include a pluggable connection.

In some embodiments, the connection interface may be further configured to make one end of an electrode transmission line in a pluggable connection with the host. The other end of the electrode transmission line may be connected with one end of the electrode via an electrical signal, and the other end of the electrode may be near to the target area.

In some embodiments, the output module may include an alarm unit. In response to determining that the monitoring information exceeds a preset threshold, the alarm unit may an alarm prompt.

In some embodiments, the output module may be disposed on the host.

In some embodiments, the host may be fixable on a surgical bed.

In some embodiments, the electrode may include a pin type electrode of which a length ranges from 4 cm to 10 cm.

In some embodiments, the host may further include a threshold adjustment unit connected to the signal processing module via an electrical signal. The threshold adjustment unit may be configured to adjust a threshold in advance.

In some embodiments, a maximum size of the host of the monitoring device may be less than 50 mm.

Embodiments of the present disclosure provides a method for controlling a threshold of a monitoring device. The method may include: setting a threshold of the monitoring device associated with the electromyographic signal; placing an electrode of the information acquisition module to the target area. The electrode may be configured to acquire the electromyographic signal and transmit the electromyographic signal to the signal processing module; and starting the monitoring device to output, based on the threshold, the monitoring information corresponding to the electromyographic signal.

In some embodiments, the monitoring device may include a first adjustment member and a second adjustment member for setting a threshold.

In some embodiments, setting the threshold of the monitoring device may include: triggering the first adjustment member to increase the set threshold; or triggering the second adjustment member to decrease the set threshold.

In some embodiments, setting the threshold of the monitoring device may include: setting the threshold by a text input box of the monitoring device.

### BRIEF DESCRIPTION OF THE DRAWINGS

The present disclosure is further described in terms of exemplary embodiments. These exemplary embodiments are described in detail with reference to the drawings. These embodiments are not limiting, and in these embodiments, like reference numerals represent similar structures, and wherein:

FIG. 1 is a schematic diagram of a nerve monitoring system according to some embodiments of the present disclosure;

FIGS. 2 and 3 are schematic diagrams of an exemplary nerve monitoring device according to some embodiments of the present disclosure, respectively;

FIG. 4 is a circuit diagram of an exemplary filter unit according to some embodiments of the present disclosure;

FIG. 5 is a circuit diagram of an exemplary amplification unit and an exemplary analog to digital converter according to some embodiments of the present disclosure;

FIG. 6 is a schematic diagram of an exemplary circuit module of a nerve monitoring device according to some embodiments of the present disclosure;

FIG. 7 is a schematic diagram of an exemplary application scenario of a nerve monitoring system according to some embodiments of the present disclosure;

FIG. 8A is a schematic diagrams of an exemplary signal acquisition module in the nerve monitoring system of FIG. 7;

FIG. 8B is a schematic diagrams of an exemplary nerve detecting device in the nerve monitoring system of FIG. 7;

FIG. 9 is a schematic diagram of an exemplary application scenario of a nerve monitoring system according to some embodiments in the present disclosure;

FIGS. 10A and 10B are schematic diagrams of an exemplary signal acquisition module in the nerve monitoring system of FIG. 9;

FIG. 11 is a schematic diagram of an exemplary application scenario of a nerve monitoring system according to some embodiments of the present disclosure;

FIG. 12 is a schematic diagram of an exemplary stimulation module in the nerve monitoring system of FIG. 11;

FIG. 13 is a cross-sectional view showing an exemplary nerve detecting device according to some embodiments of the present disclosure;

FIG. 14 is a schematic diagram of an exemplary nerve detecting device according to some embodiments of the present disclosure; and

FIG. 15 is a schematic diagram of an exemplary connection structure between a probe and a sleeve according to some embodiments of the present disclosure.

### DETAILED DESCRIPTION

In order to illustrate the technical solutions related to the embodiments of the present disclosure, a brief introduction of the drawings referred to in the description of the embodiments is provided below. Obviously, drawings described below are only some examples or embodiments of the present disclosure. Those having ordinary skills in the art, without further creative efforts, may apply the present disclosure to other similar scenarios according to these drawings. Unless obviously obtained from the context or the context illustrates otherwise, the same numeral in the drawings refers to the same structure or operation.

It should be understood that “systems”, “devices”, “unit”, and/or “modules” used herein are used to distinguish different components, elements, parts, or assemblies in different levels. However, if other words can achieve the same purpose, the words can be replaced by other expressions.

As shown in the present specification and claims, the singular forms “a,” “an,” and “the” include plural forms as well unless the content clearly indicates otherwise. In general, the terms “comprise,” “comprising,” “include,” and/or “including” when used in this disclosure, specify the presence of stated steps and elements, and these steps and elements do not constitute an exclusive listing. The methods or devices may also include other steps or elements.

The flowcharts used in the present disclosure illustrate operations that systems implement according to some embodiments of the present disclosure. It should be understood that the preceding or following operations may be implemented not in order. Conversely, the operations may be implemented in an inverted order, or simultaneously. Moreover, one or more other operations may be added to the flowcharts. One or more operations may be removed from the flowcharts.

Embodiments of the present disclosure may be applied to surgery for helping relevant personnel (e.g., medical staff, family members of patients, etc.) to identify, position, and/or protect nerves. Although the present disclosure mainly describes human nerves as an example, it should be noted that the principles of the present disclosure can also be applied to animal nerves. It should be understood that the application scenarios of a nerve monitoring system of the present disclosure are only some examples or embodiments of the present disclosure, and the nerve monitoring system may also be applied to other similar scenarios based on these figures for those skilled in the art without paying creative labor.

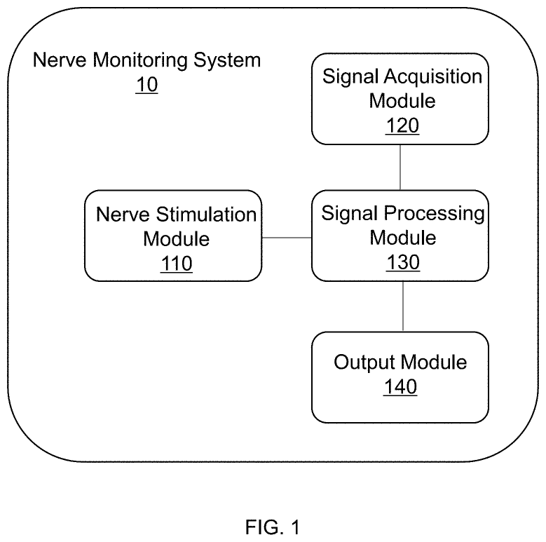

FIG. 1 is a schematic diagram of an exemplary nerve monitoring system according to some embodiments of the present disclosure.

As shown in FIG. 1, the nerve monitoring system may include a nerve stimulation module 110, a signal acquisition module 120, a signal processing module 130, and an output module 140.

The nerve stimulation module 110 may include a nerve detecting device. The nerve detecting device may be used to stimulate a nerve of a target area by stimulating a current. In some embodiments, the target area refers to a diseased area of a subject on which surgery is to be performed. An electromorphic signal generated by the stimulated nerve may be transmitted through the tissue and may be received by the signal acquisition module 120 connected to the target area. The signal acquisition module 120 may transmit the received electromyographic signal to the signal processing module 130. The signal processing module 130 may process the received electromyographic signal to control the output module 140 connected to the signal processing module 140 to output nerve monitoring information corresponding to the electromyographic signal. The nerve monitoring information may include information indicating whether a nerve suffers from a stimulation satisfying a preset condition. In some embodiments, the preset condition may include that a voltage peak of an electromyographic signal exceeds a set voltage threshold.

In some embodiments, the voltage threshold may be 100 μV, or may be adjusted according to different subjects or application scenarios. Specifically, different voltage thresholds may be set according to sensitivities of nerves for different patients. For example, if a nerve of a patient is relatively sensitive, the voltage threshold may be decreased, and if a nerve of a patient is relatively insensitive, the voltage threshold may be increased. Different voltage thresholds may be set according to a diseased portion of a patient. For example, a voltage threshold of a throat nerve may be lower than a voltage threshold of a foot nerve. When a voltage is higher than the voltage threshold, it may be determined that the target area has been largely stimulated, that is, the target area includes neural tissue.

In some embodiments, an operator may operate the nerve detecting device to contact the tissue of the target area, and thus determine whether a stimulated portion is a nerve according to the nerve monitoring information output from the output module 140. Based on this, a surgeon may identify and position a nerve by operating the nerve detecting device, thereby avoiding injury to nerves during surgery.

In some embodiments, the nerve may be stimulated for a plurality of reasons. In some embodiments, the nerve may be stimulated by a particular nerve stimulation module, such as the nerve detecting device described above. In some embodiments, a surgeon may bring stimulation to the nerve during the routine surgical operation. For example, a surgeon may directly touch a nerve or indirect affect a nerve when performing an operation on the tissue, causing stimulation to the nerve, cold/hot water to clean tissue may also cause stimulation for related nerves, etc. Therefore, the surgeon may determine whether the nerve suffers from a stimulation satisfying a preset condition based on the nerve monitoring information output from the output module 140, thereby avoiding injury to the nerve caused by some surgical operations. For example, it may be determined whether there is a nerve that touched based on the nerve monitoring information, thereby determining a position of the nerve. It may be determined a degree of a nerve stimulated by the water temperature to clean the current tissue according to the nerve monitoring information, such that the water temperature can be adjusted to an appropriate temperature to make a patient feel comfortable.

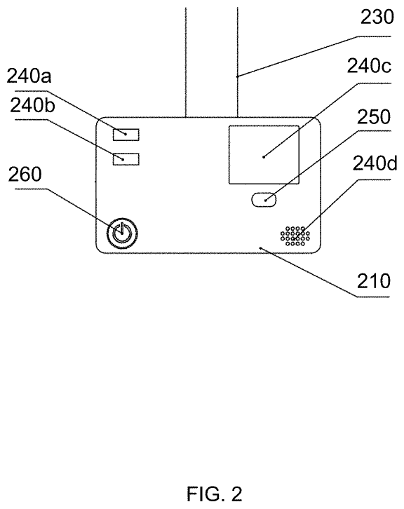

Specific implementation of the modules and devices of the nerve monitoring system and the integration modes between the modules may refer to FIGS. 2-15 and descriptions thereof. It should be understood that the implementation and principles of the same modules, devices, structures, etc. with the same name in different drawings may be referred to each other.

FIGS. 2 and 3 are schematic diagrams of an exemplary nerve monitoring device according to some embodiments of the present disclosure, respectively.

In some embodiments, the nerve monitoring system may include a nerve monitoring device and a nerve stimulation device (including a nerve detecting device). The nerve monitoring device may be configured to detect an electromyographic signal in a target area stimulated by the nerve detecting device. In some embodiments, the nerve monitoring system may only include a nerve monitoring device used to detect an electromyographic signal generated when a nerve suffers from an external stimulation. In some embodiments, the external stimulation refers to a stimulation that a body receives from an external environment, which may cause the body to generate an electromyographic signal. The external stimulation may include a stimulation generated by the nerve detecting device, or a stimulation generated by a normal operation of a doctor according to the pathology.

Referring to FIGS. 2 and 3, in some embodiments, the nerve monitoring device may include a host 210 and a signal acquisition module.

In some embodiments, the host 210 may include a signal processing module. A signal acquisition module may be connected to the signal processing module via an electrical signal to transmit an acquired electromyographic signal.

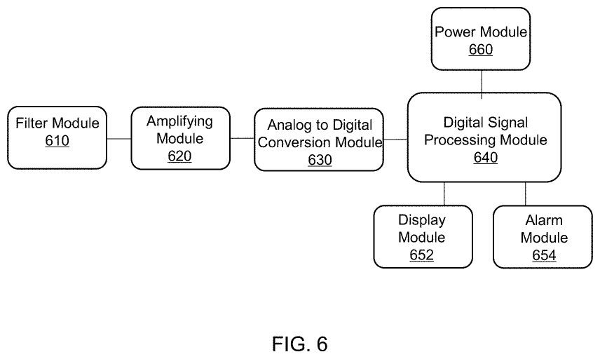

In some embodiments, as shown in FIGS. 2 and 3, the signal acquisition module 120 may include an electrode for acquiring an electromyographic signal, the electromyographic signal may be transferred to the signal processing module 130. In some embodiments, at least two electrodes may be used to acquire the electromyographic signal since the electromyographic signal is a voltage differential signal. In some embodiments, a material of the electrode may include a high-conductive material such as medical stainless steel, high conductive rubber. In some embodiments, the material of the electrode may include a material having a low elastic modulus. For example, an elastic modulus of the material of the electrode may be 5 MPa to 10 MPa, 6 MPa to 9 MPa, or 7 MPa to 8 MPa. Specifically, the elastic modulus of the electrode material may be 7.84 MPa. In other embodiments, the material of the electrode may be a material having a high elastic modulus. For example, the elastic modulus of the material of the electrode may be 2×10⁴ MPa. In some embodiments, the material of the electrode may have a resistivity of 10⁻⁸ Om to 10⁻⁷ Ωm. The material of the electrode may be 1.65×10⁻⁸ Ωm. In some embodiments, the electrode may include a pin type electrode 230 as shown in FIG. 2, a patch type electrode 230 as shown in FIG. 3, or a pin-patch type electrode (not shown). In some embodiments, a length of the pin type electrode may range from 1 cm to 20 cm, from 2 cm to 15 cm, from 4 cm to 10 cm, from 5 cm to 9 cm, or from 6 cm to 8 cm. In some embodiments, the electrode and the signal processing module 130 may be in an electrical signal connection in a wire or wireless manner. In some embodiments, when the electrode is connected to the signal processing module 130 in a wireless manner to transmit an electromyographic signal, a wireless transmission module may be disposed between the electrode and the signal processing module. More description about the electrode may refer to FIGS. 7 to 12 and related descriptions thereof.

In some embodiments, the host 210 may be provided with a connection interface. The connection interface may be configured to connect the electrode directly to the signal processing module 130 via an electronic signal. In some embodiments, the connection interface may be configured to connect the electrode directly to a main board (not shown) of the host. Thus, not only space can be saved, but the noise introduced during the electromyographic signal transmission can be effectively reduced and the stability of signal transmission can be improved. In some embodiments, the main board of the host may be understood as a circuit board integrating each circuit module of the host.

In some embodiments, the output module may be at least configured to output nerve monitoring information, which can indicate whether a nerve suffers from a stimulation satisfies a preset condition and/or a stimulation degree of a nerve. The preset condition may include a voltage peak of an electromyographic signal exceeds a set voltage threshold. In some embodiments, the output module may include a sound prompt unit, a light prompt unit, a display unit, or the like, or any combination thereof. For example, the output module may include an alarm unit, such as a light prompt unit 240a, a light prompt unit 240b, a display unit 240c, and/or a sound prompt unit 240d shown in FIGS. 2 and 3. Accordingly, the nerve monitoring information may be in the form of sound, an image, lighting, or the like, or any combination thereof. In some embodiments, the nerve monitoring information may include a parameter value of the electromyographic signal corresponding to a nerve detected in a target area. In some embodiments, the nerve monitoring information may include alarm information indicating that a stimulation of a nerve in the target area reaches the preset condition. Medical personnel or family member(s) of a patient may confirm that a nerve suffers from a stimulation larger than a preset threshold.

In some embodiments, the alarm information may be presented by broadcasting a voice through a speaker, a buzzer scream, a light emitting diode (LED) light, a display, etc. Accordingly, the output unit may include an alarm unit in one or more of a speaker, a buzzer, an LED, a display unit, etc. The display unit may output the alarm information by displaying words, images, and/or a pop-up window. Further, the alarm information may be easier to be concerned by increasing a size of the words and images, displaying a special symbol (such as an exclamation mark), displaying an eye-catching color, etc., or any combination thereof.

In some embodiments, the nerve monitoring information may include a waveform and/or a voltage peak of an electromyographic signal displayed by the display unit, or a voltage peak of an electromyographic signal broadcasted by the sound prompt unit. Medical staff may determine whether a nerve suffers from a stimulation satisfying the preset condition by viewing the displayed waveform and/or voltage peak, or by listening to the broadcasted voltage peak, which may refer to the description about the signal processing module.

In some embodiments, the light prompt unit may present alarm information by changing a light to be on or off, light flashing, setting light colors, increasing the lighting brightness, or the like. For example, a light is turned off, indicating that a nerve does not suffer from a stimulation satisfying the preset condition, and a light is on, indicating that a nerve suffers from a stimulation satisfying the preset condition. As another example, a light is on, indicating that a nerve does not suffers from a stimulation, a light is flashing, indicating that a nerve suffers from a stimulation satisfying the preset condition. As a further example, a green light may indicate that a nerve does not suffer from a stimulation satisfying the preset condition, and a red light may indicate that a nerve suffers from a stimulation satisfying the preset condition. As still another example, a low-brightness light may indicate that a nerve does not suffer from a stimulation satisfying the preset condition, and a high-brightness light may indicate that a nerve suffers from a stimulation satisfying the preset condition.

In some embodiments, the output module may further include an operation information prompt unit for outputting operation information indicating an operation state of the nerve monitoring device. The operation information may include information indicating a good condition of an electrical connection. In some embodiments, a signal processing unit may determine the good condition of the electrical connection by detecting whether the electromyographic signal is received. When the signal processing module does not detect the electromyographic signal, there may be a connection problem between the signal acquisition module 120 and the signal processing module 130 and/or the signal acquisition module 120 and the tissue. For example, the electrode for signal acquisition falls off from the target area during surgery, failing to acquire the electromyographic signal. After knowing that the information indicating that the electrical connection is poor, the relevant personnel may check whether there are connection problems between the signal acquisition module 120 and the signal processing module 130 and between the signal acquisition module 120 and the tissue and adjust the connection in time. For example, if the relevant personnel found the electrode for signal acquisition falls off from the target area, the electrode may be re-connected to the target area and secure it. In some embodiments, with reference to the description above, the operation information may be presented by the light prompt unit, the display unit, or the sound prompt unit, or the like. Taking the light prompt unit as an example, in some embodiments, with reference to FIGS. 2 and 3, different light prompt units may be used to present different information. For example, the light prompt unit 240a may present information indicating that an electrical connection is good and the lighting prompt unit 240b may present the nerve monitoring information, or the light prompt unit 240a may present the nerve monitoring information and the lighting prompt unit 240b may present the information indicating that an electrical connection is good. In some embodiments, detailed description about the light prompt unit presenting information indicating the electrical connection is good may refer to the aforementioned description about the light prompt unit presenting the nerve monitoring information. For example, the light is on, indicating the connection is good, and light flashing indicates the connection is poor.

In some embodiments, the output module (for example, the light prompt unit 240a, the light prompt unit 240b, the display unit 240c, and the sound prompt unit 240d shown in FIGS. 2 and 3) may be disposed on the host 210. By disposing the output module on the host 210, there may be little effect on the overall volume of the nerve monitoring device. In this way, the surgeon may receive the information output by the output module at a close distance by placing the relatively small nerve monitoring device next to a surgical object during the surgery, which greatly improves the convenience of using the nerve monitoring device. Referring to FIGS. 2 and 3, when the output module is integrated on the host, in some embodiments, the electrode may be in a direct connection with the host. The direct connection may include a pluggable direct connection, or an unpluggable direct connection. In some embodiments, the electrode may also be connected to a host integrating with the output module by an electrode transmission line or wireless transmission. For example, an interface for connecting to the electrode, a printed signal processing circuit, and an output circuit may be disposed on the same main board, and an input voltage of the electrode may be displayed by an output circuit.

In some embodiments, the host 210 may be provided with a power source (not shown) and a power switch 260.

In some embodiments, the host 210 may include a connection interface directly connected to the electrode, or a connection interface indirectly connected to the electrode. The host may include a host integrating with the output module, or without the output module. In some embodiments, the electrode may be connected to the connection interface through a transmission line, or via a set of wireless transceiver modules. In some embodiments, the transmission line and/or wireless transceiver module may be directly connected to the signal processing unit without passing through the connection interface. In some embodiments, the connection interface may be pluggable. Detailed description about the specific implementation of signal transmission through the transmission line/wireless transceiver modules may refer to FIGS. 7 to 12 and related description thereof.

In some embodiments, the signal processing module may include a filter unit, an amplifying unit, an analog to digital converter, and a digital signal processor. The filter unit may be used to filter out noise carried by the electromyographic signal during the transmission process. The amplification unit may be used to amplify an amplitude of the electromyographic signal to the level of the analog-to-digital converter. The analog to digital conversion unit may be used to convert the amplified electromyographic signal into a digital signal and then transmit the digital signal to a digital signal processor for processing.

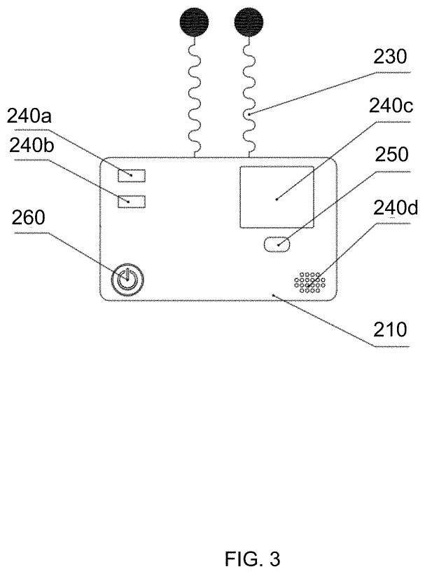

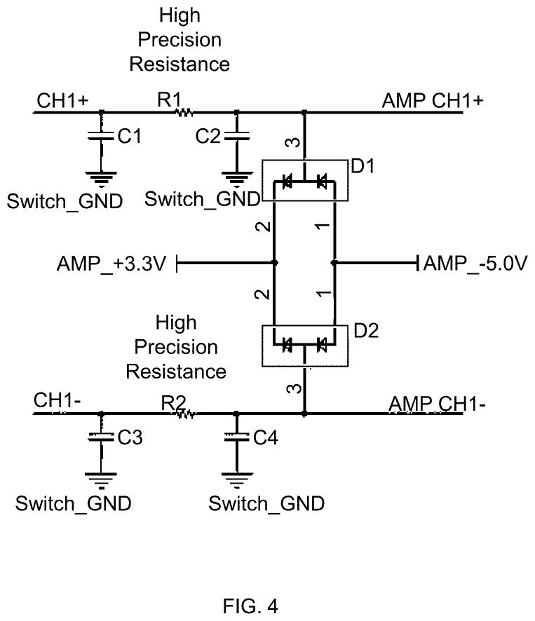

FIG. 4 is a circuit diagram of an exemplary filter unit according to some embodiments of the present disclosure. FIG. 5 is a circuit diagram of an exemplary amplification unit and an exemplary analog to digital converter according to some embodiments of the present disclosure.

As shown in FIG. 4, one end of each electrode of a pair of electrodes may be connected to a target area and the other end may be connected to an input end (on the left of FIG. 4) of a filter circuit. In some embodiments, the connection between the one end of each electrode and the target area may be implemented by making the one end of each electrode approach the target area. In some embodiments, making the one end of each electrode approach the target area may include inserting the one end of each electrode into the skin tissue, attaching the one end of each electrode on the skin surface, or making the one end of the electrode close to the skin surface but not in contact with the skin. In some embodiments, a first differential signal (i.e., an electromyographic signal) including CH1+ and CH1− acquired by the pair of electrodes from the target area (one electrode acquires CH1+, the other electrode acquires CH1−) may be transmitted to the filter circuit, and a second differential signal including AMP CH1+ and AMP CH1− may be obtained after filtering. The second differential signal may be input to an operational amplifier in the exemplary amplification unit shown in FIG. 5. As shown in FIG. 5, AMP CH1 may be input to a same phase input end +IN (pin 4) in the operational amplifier, AMP CH1− may be input to a reverse phase input end −IN (pin 1). An output end OUT (pin 7) of the operational amplifier may output an amplified difference signal of CH1+ and CH1− (i.e., an amplified electromyographic signal). The amplified electromechanical signal may be transmitted to an input end VinL (pin 1) of the analog-to-digital converter, and the analog-to-digital converter may convert the received (amplified) electromyographic signal into a digital signal and be output by the output end DOUT (pin 12). The output digital signal may be transmitted to the digital signal processor in the next level.

In some embodiments, the voltage amplitude range of the electromyographic signal acquired by the signal acquisition module may be from 5 μV to 70 mV, from 5 μV to 60 mV, from 5 μV to 50 mV, from 5 μV to 40 mV, from 5 μV to 30 mV, from 5 μV to 20 mV, from 5 μV to 10 mV, from 5 μV to 5 mV, or from 5 μV to 1 mV. In some embodiments, the magnification of the amplification unit may be 20 to 100 times. In some embodiments, the analog to digital converter may use a 24-bit output analog-to-digital conversion chip to achieve a higher resolution on the voltage of the electromyographic signal.

In some embodiments, the processing of the electromyographic signal by the digital signal processor may include restoring the waveform of the electromyographic signal according to the received digital signal, and/or determining the voltage peak of the electromyographic signal. The waveform and voltage peak of the electromyographic signal may be regarded as nerve monitoring information. Since the electromyographic signal is significantly changed before or after a certain degree of a stimulation to the nerve, the peak voltage of the electromyographic signal may be relatively high. By setting an appropriate voltage threshold and comparing the peak voltage of the electromyographic signal with the voltage threshold, it is possible to identify whether the nerve suffers from the stimulation satisfying the preset condition according to the comparison result. Based on this, in some embodiments, the digital signal processor may directly control the display unit to display the waveform and/or the voltage peak of electromyographic signal. In some embodiments, the digital signal processor may obtain a recognition result of whether the nerve suffers from the stimulation satisfying the preset condition by comparing the voltage peak of the electromyographic signal with the preset voltage threshold, thereby controlling the output unit to output nerve monitoring information corresponding to the recognition result. Detailed description about the specific presentation for nerve monitoring information is described above, and herein are omitted.

Since the peak voltage of the electromyographic signal generated by the stimulated nerve is affected by some factors such as the type of nerves, the body, the voltage threshold may be set according to the factors before the voltage peak and the preset voltage threshold of the electromyographic signal are compared. To this end, in some embodiments, the signal processing module may pre-adjust the voltage threshold for identifying whether the nerve suffers from the stimulation satisfying the preset condition according to a user instruction. Exemplary methods for adjusting the voltage threshold may be described following.

In some embodiments, the host 210 may further include a threshold adjustment unit connected to the signal processing module, and the threshold adjustment unit may be disposed on the host 210. The threshold adjustment unit may be configured to generate a user instruction to adjust the voltage threshold according to a user action and transmit the user instruction to the signal processing module. A user may adjust the voltage threshold by operating the threshold adjustment unit. In some embodiments, an operation mode of the threshold adjustment unit may include pressing a button (see the button 250 in FIGS. 2 and 3), rotating a button, sliding a block, etc. In some embodiments, the threshold adjustment unit may include multiple buttons when the operation mode of the threshold adjustment unit includes pressing a button. For example, the threshold adjustment unit may include a first button and a second button. The first button may be configured to generate an instruction for increasing the current voltage threshold to a preset value. The second button may be configured to generate an instruction for reducing the current voltage threshold to a preset value. As another example, the threshold adjustment unit may include a reset button for generating an instruction for resetting the current voltage threshold to a preset default value and an adjustment button for generating an instruction for changing the current voltage threshold in a preset manner. In some embodiments, when the output module includes a display unit, the display unit may be used to display a configuration interface of the voltage threshold. Further, the display unit may be in a touch type and configured to generate a user instruction according to the user's touch operation and send the user instruction to the signal processing module, so that the signal processing module can adjust the voltage threshold according to the received user instruction.

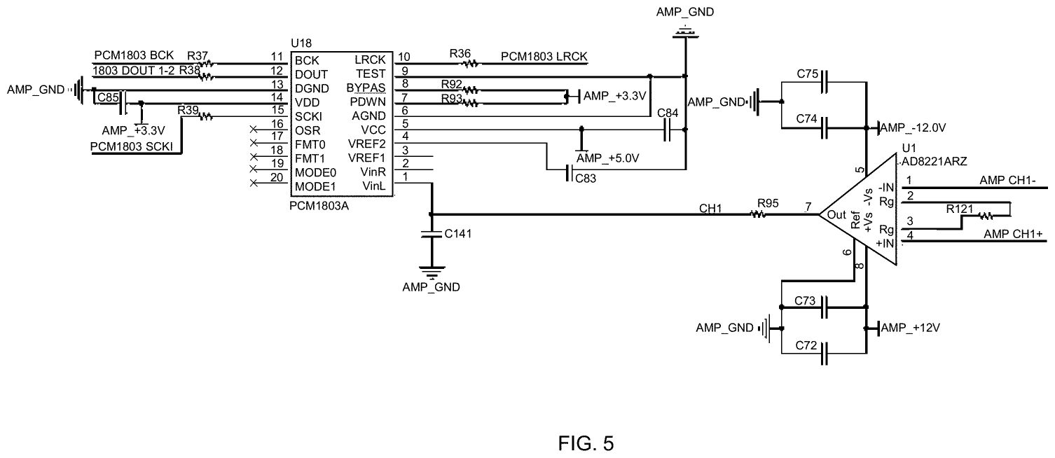

FIG. 6 is a schematic diagram of an exemplary circuit module of a nerve monitoring device according to some embodiments of the present disclosure.

As shown in FIG. 6, the nerve monitoring device may include a filter module 610, an amplifying module 620, an analog to digital conversion module 630, a digital signal processing module 640, a display module 652, an alarm module 654, and a power module 660. One end of an electrode of acquiring the electromyographic signal may be connected to a target area, and the other end may be connected to an input end of the filter module 610. An output end of the filter module 610 may be connected to an input end of the amplifying module 620. The analog to digital conversion module 630, the display module 652, the alarm module 654, and the power module 660 may be connected to the digital signal processing module 640. It should be understood that the filter module 610, the amplifying module 620, the analog to digital conversion module 630, the digital signal processing module 640, the display module 652, the alarm module 654, and the power module 660 may be implemented in the aforementioned filter unit, the amplifying unit, the analog to digital conversion unit, the digital signal processing unit, the display unit, the alarm unit, and the power supply, respectively.

Since the nerve monitoring device without the nerve stimulation module integrated therein may be designed as an integrated device in a relatively small volume, it may be placed close to the surgical object and the surgeon when used, such as on an operating table. Not only does it cause little hindrance to the surgical operation, but also facilitates the surgeon to operate the nerve monitoring device closely. In some embodiments, the nerve monitoring device may be secured, by a fixing member, to a subject around a patient, such as a surgical bed, a sheet, a quilt, or the like. In some embodiments, the fixing member may include one or more of a clip, a tape, a rubber band, or the like. In some embodiments, a size of the nerve monitoring device may refer to a size of the host 210. Further, the size of the nerve monitoring device may refer to a size of the host with the signal processing module and the output module integrated therein. In some embodiments, a maximum size (e.g., at least one of the length, width, height) of the nerve monitoring device may be less than 300 mm, 200 mm, 80 mm, 60 mm, or 50 mm. For example, in some embodiments, the size of the nerve monitoring device may reach 50 mm*30 mm*30 mm.

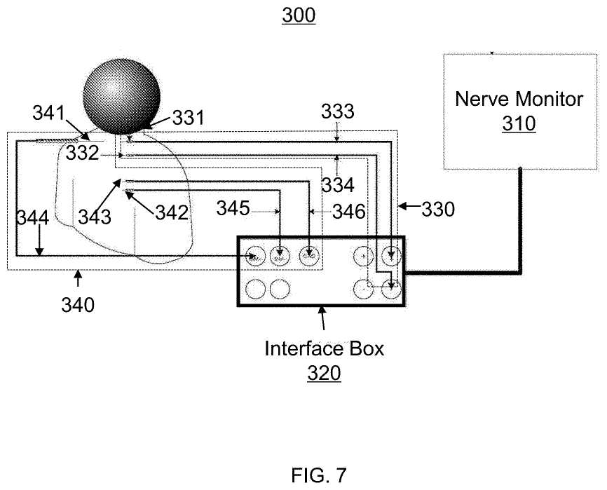

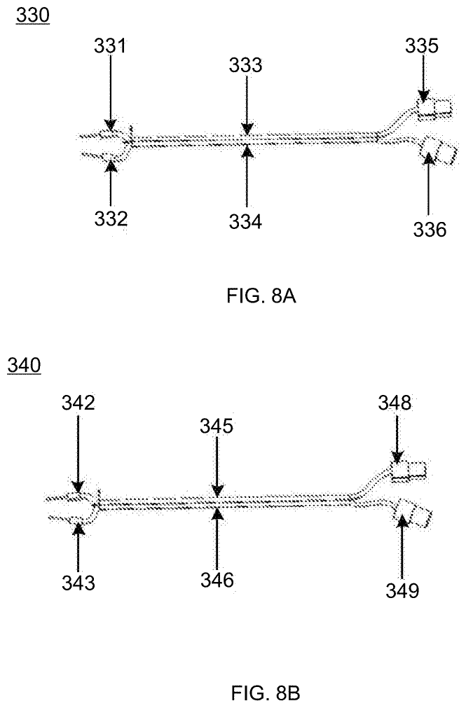

Please refer to FIGS. 7, 8A, and 8B. FIG. 7 is a schematic diagram of an application scenario of a nerve monitoring system 300 according to some embodiments of the present disclosure. FIG. 8A is a schematic diagram of a signal acquisition module 330 in the nerve monitoring system 300. FIG. 8B is a schematic diagram of a nerve detecting device 340 in the nerve monitoring system 300.

As shown in FIG. 7, the nerve monitoring system 300 may include a nerve monitor 310 and an interface box 320. A signal acquisition module 330 and a nerve detecting device 340 may be connected to a signal processing module through the interface box 320. The nerve monitor 310 may integrate with a stimulation signal generating device and a signal processing module. Description about the specific functions of the stimulation signal generating device and signal processing module may refer to FIG. 1 and related description. In some embodiments, the signal processing module may adjust stimulation parameter(s) of a stimulation module. In some embodiments, the signal processing module may be displayed by displaying a setting interface of stimulation parameter(s) through the display unit. Description about the adjustment of the signal processing module on the stimulation parameter(s) and cooperation with the display unit may refer to FIG. 1 and related description.

The signal acquisition module 330 may form an electric circuit required to acquire an electromyographic signal between the human body and the interface box 320. In some embodiments, as shown in FIGS. 7, 8A, and 8B, the signal acquisition module 330 may include a first electrode 331, a second electrode 332, a first electrode line 333, a second electrode line 334, a first electrode connector 335, and a second electrode connector 336. In other embodiments, more than two sets of electrodes, electrode lines, and electrode connectors may be employed, such as 3 sets, 4 sets, 5 sets, 6 sets or more. When surgery, the first electrode 331 and the second electrode 332 may be connected to a target area with a distance of about 1 cm, the first electrode connector 335 and the second electrode connector 336 may be connected to one pair of positive and negative electrode interfaces of the interface box 320, so that an electric circuit is formed.

The nerve detecting device 340 may form an electric circuit required to acquire the stimulation signal between the human body and the interface box 320. In some embodiments, as shown in FIGS. 7, 8A, and 8B, the nerve detecting device 340 may include a probe 341, a stimulation electrode 342, a ground electrode 343, a probe connection line 344, a stimulation electrode line 345, a ground electrode line 346, a probe connector (not shown), a stimulation electrode connector 348, and a ground electrode connector 349. When surgery, the stimulation electrode 342 and the ground electrode 343 may be connected to a target area with a distance of about 7 cm, the probe connector and the stimulating electrode connector 348 may be respectively connected to a pair of positive and negative electrode interfaces of the interface box 320, the ground electrode connector 349 may be connected to the ground interface of the interface box 320, such that an electric circuit is formed when the probe 341 contacts with the human body.

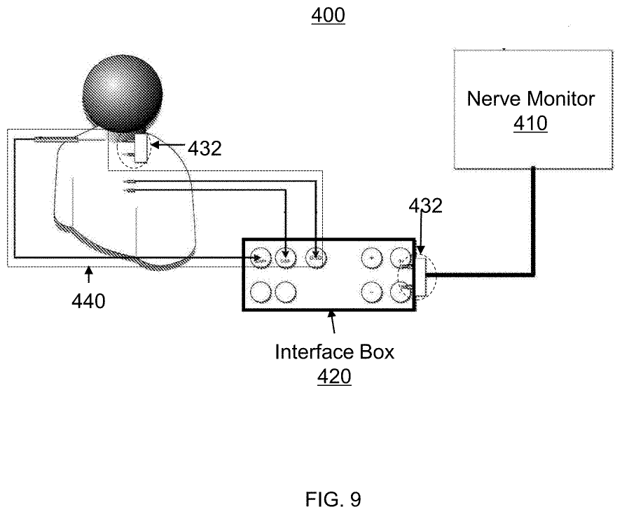

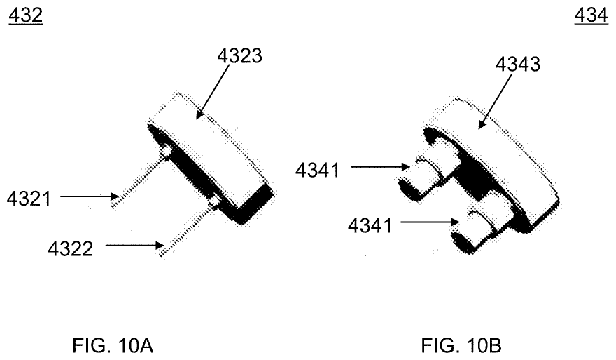

Please refer to FIGS. 9, 10A, and 10B. FIG. 9 is a schematic diagram of an application scenario of a nerve monitoring system 400 according to some embodiments in the present disclosure. FIGS. 10A and 10B are schematic diagrams of a signal acquisition module 430 in the nerve monitoring system 400.

As shown in FIG. 9, the nerve monitoring system 400 may also include a nerve detecting device 440, a nerve monitor 410, and an interface box 420. Description about the specific function of the nerve detecting device, the nerve monitor, and the interface box may refer to the FIGS. 7, 8A, and 8B, and related description thereof.

Different from the signal acquisition module 230 in the wired transmission mode shown in FIGS. 7, 8A, and 8B, the signal acquisition module 430 in the nerve monitoring system 400 may transmit the acquired electromyographic signal to the interface box 420. In some embodiments, the signal acquisition module 430 may include an electromyographic signal transmitting device 432 and an electromyographic signal receiving device 434 as shown in FIGS. 9, 10A, and 10B. As shown in FIGS. 10A and 10B, the electromyographic signal transmitting device 432 may include a first electrode 4321, a second electrode 4322, a wireless transmitting module 4323 connected to the first electrode 4321 and the second electrode 4322. The electromyographic signal receiving device 434 may include a wireless receiving module 4343, and a first electrode connector 4341 and a second electrode connector 4342 connected to the wireless receiving module 4343. In other embodiments, more than one set of electrode pairs, wireless transmitting and receiving modules, and connector pairs may be used, such as 2 sets, 3 sets, or more. When surgery, the first electrode 4321 and the second electrode 4322 may be connected to a diseased region of a human, and the first electrode connector 4341 and the second electrode connector 4342 may be respectively connected to a pair of positive and negative electrode interfaces of the interface box 420, so that an electromyographic signal collected by the first electrode 331 and the second electrode 33 may be sent to the interface box through a pair of wireless transmitting and receiving modules. In some embodiments, through appropriate circuit improvement, the first electrode connector 335 and the second electrode connector 336 may be combined into an electrode connector connected to a control loop of the interface box. By wirelessly transmitting the acquired electromyographic signal, the inconvenience caused by the transmission line can be avoided.

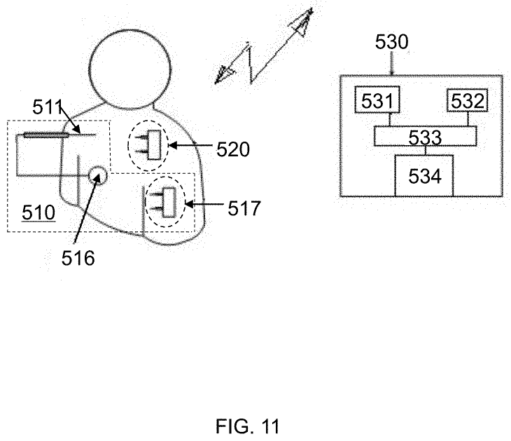

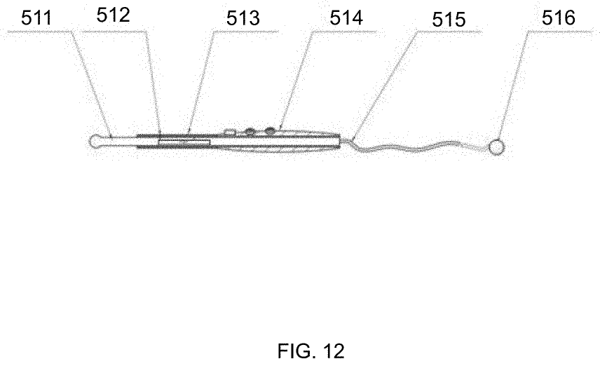

Please refer to the FIGS. 11 and 12. FIG. 11 is a schematic diagram of the application scenario of a nerve monitoring system 500 according to some embodiments of the present disclosure, and FIG. 12 is a schematic diagram of a stimulation module 510 in the nerve monitoring system 500.

As shown in FIG. 11, the nerve monitoring system 500 may include a signal acquisition module 520 and a host 530. A stimulation module 510 may include a pair of electrodes for acquiring an electromyographic signal and a stimulation signal wireless transmitting module (i.e., a stimulation signal transmitting portion 517 shown in FIG. 11). The signal acquisition module 520 may include a pair of electrodes for collecting an electromyographic signal and an electromyographic signal wireless transmitting module connected to the pair of electrodes. The host 530 may be integrated with a stimulation signal wireless receiving module 531 and an electromyographic signal wireless receiving module 532 When a stimulation signal wireless transmitting module is wirelessly connected to a stimulation signal wireless receiving module 531 or an electromyographic signal wireless transmitting module is wirelessly connected to electromyographic signal wireless receiving module 532, the wireless transmission of the stimulation signal/electromyographic signal can be realized between the human body and the host 530, which can avoid the inconvenience caused by the transmission line.

In some embodiments, the host 530 may further include a signal processing module 533 and an output module 534. The stimulation signal wireless receiving module 531, the electromyographic signal wireless receiving module 532, and the output module 534 may be connected to the signal processing module 533 to transmit the received electromyographic signal/stimulation signal to the signal processing module 533. In some embodiments, the stimulation signal wireless receiving module 531 and the electromyographic signal wireless receiving module 532 may be two separate wireless communication modules, or a wireless communication module. The signal processing module 533 may be used to process the received stimulation signal/electromyographic signal to control the output module 534 to output output information related to the stimulation signal/electromyographic signal.

In some embodiments, the stimulation module 510 may be designed as a hand-held structure as shown in FIG. 12. As shown in FIG. 12, the hand-held stimulation module 510 may include a probe 511, an insulating layer 513, a handle 514, an electrode connection line 515, an electrode module 516, a stimulation signal generating device (not shown), a stimulating signal wireless transmitting module (not shown). One end of the probe 511 may be fixed in the handle 514. The probe 511 covering an insulating layer 513 may be located at one end of the handle. The electrode module 516 may include a stimulating electrode and a ground electrode. In some embodiments, the insulating layer 513 may include a heat shrinkable sleeve or an insulating coating. One end of the electrode connection line 515 may be connected to the probe 511, and the other end may be connected to the electrode module 516.

The stimulation signal generating device may be configured to generate a stimulation current. Due to individual differences, different surgical objects may have different sensitivity to the stimulation current. That is, for different surgical objects, values and/or durations of the stimulation current that can cause significant changes in the electromyographic signal may be different. For this reason, in some embodiments, the values and/or durations of the output current of the electromyographic signal generating device may be adjusted. Specifically, the adjustment switch and/or duration adjustment can be achieved by a connecting the stimulation signal generating device and disposed on the handle 514. In some embodiments, the current values and/or durations may be adjusted by an adjustment switch which is connected to the stimulation signal generating device and set on the handle 514. During the surgery, the medical staff may continuously, quickly, conveniently and accurately adjust the current value displayed on the handle 514 during the stimulation operation according to the needs of the surgery.

The stimulation signal wireless transmitting module may be used to transmit the stimulation signal generated by the stimulation signal generating device to the stimulation signal wireless receiving module 531 of the host 530.

In some embodiments, the stimulation signal generating device and the stimulation signal wireless transmitting module may be in a split structure or an integral structure. For the integral structure, a structural member 512 including the stimulation signal generating device and the stimulation signal wireless transmitting module may be mounted in the handle 514 as shown in FIG. 12. In some embodiments, if the structural member 512 is too large, the structural member 512 may be mounted in the electrode module 516 and placed on a patient. In some embodiments, for the split structure, one of the stimulation signal generating device and the stimulation signal wireless transmitting module may be located in the handle 514, and the other may be located in the electrode module 516.

In some embodiments, the stimulation module 510 may transmit current operating state information (e.g., a value or duration of the stimulation current) to the stimulation signal wireless receiving module 532 of the host 530 by the stimulation signal wireless transmitting module.

In some embodiments, when the output module 533 includes a display unit, the signal processing module 533 may only control the display unit to display an effective electromyographic signal. The effective electromyographic signal refers to an electromyographic signal received by the electromyographic signal wireless receiving module when the stimulation signal wireless receiving module receives the stimulation signal generated by the stimulation signal generating device.

Some embodiments of the present disclosure may include a method for controlling a threshold of the nerve monitoring device provided in the foregoing embodiments. The method may include setting a voltage threshold of a nerve monitoring device which may output prompt information based on the voltage threshold; and placing an electrode in a target area and starting the nerve monitoring device. In some embodiments, an operator may adjust the voltage threshold by a threshold adjustment unit. Further, the threshold adjustment unit may include a first adjustment member for increasing the voltage threshold to a preset value and a second adjustment member for reducing the voltage threshold to a preset value. Accordingly, the operator may trigger the first adjustment member to increase the voltage threshold, or trigger the second adjustment member to reduce the voltage threshold. In some embodiments, the display unit of the nerve monitoring device may provide a configuration interface of the voltage threshold. In some embodiments, the display unit of the nerve monitoring device may be a touch-type and provide a configuration interface of the voltage threshold, and a user may directly enter a voltage threshold to be set by a touch operation. In some embodiments, a display unit of the nerve monitoring device may display the voltage threshold for a user by a text input box. In some embodiments, the nerve monitoring device may be fixed to a position where it is convenient for a surgeon to operate, for example, a position in a relatively close distance to a surgery object, such as a surgical bed, a sheet, and a quilt. More description about method for controlling the threshold of the nerve monitoring device may refer to the related description of the nerve monitoring device described above.

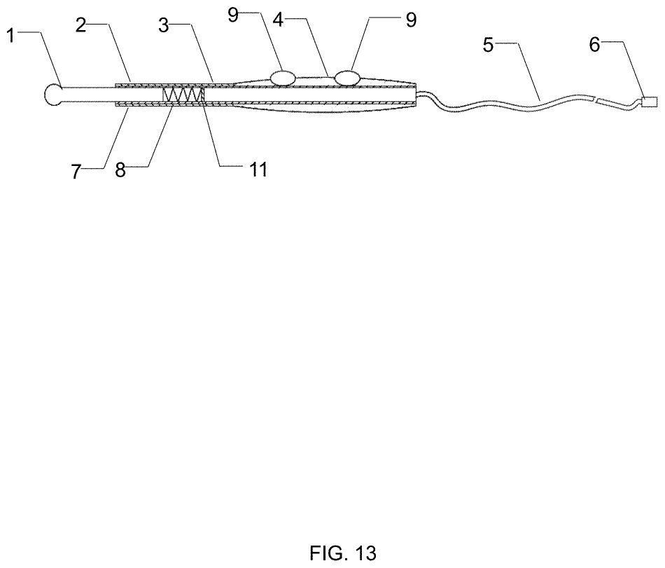

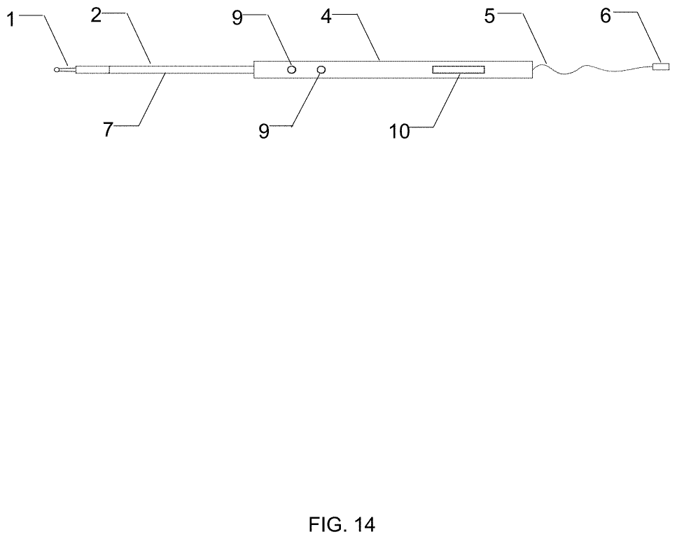

FIG. 13 is a cross-sectional view showing a nerve detecting device according to some embodiments of the present disclosure. FIG. 14 is a schematic diagram of a nerve detecting device according to some embodiments of the present disclosure.

The nerve detecting device may include a handle 4, a probe 7, and an elastic force prompt member 10. The probe 7 may be connected to the handle 4. The probe 7 may include a probe head 1, an elastic member 8, and an elastic force measuring member 11. The probe head 1 may be connected to the elastic member 8. In use, the probe head 1 may be in contact with a human body (such as a nerve, tissue, etc.), and then receive the pressure administered by the human body. The probe head 1 may transmit the pressure to the elastic member 8, and the elastic member 8 may undergo elastic deformation, causing the probe head 1 to move. Due to the elastic deformation of the elastic member, the probe head 1 is retractable, so the probe head 1 can be continuously contacted with the human body. In addition, the user may feel a resilience force when using the nerve detecting device of the present disclosure, thereby sensing the pressure exerted by the probe 7 on the human body. This allows the user to control the strength of using the nerve detecting device, and to ensure reliable contact between the probe and the nerve or tissue. The elastic force measuring member 11 may be connected to the elastic member 8 for measuring an elastic force of the elastic member 8 and converting the elastic force into an electrical signal. The elastic force prompt member 10 may be connected to the elastic force measuring member 11 to receive an electrical signal of the elastic force of the elastic member 8 generated by the elastic measuring member 11, and generate prompt information about the elastic force of the elastic member 8 based on the electrical signal. The elastic force prompt member 10 may prompt the elastic force of the elastic member in various forms including but not limited to a text, an image, a voice, or the like.

As shown in FIG. 14, the elastic force prompt member 10 may be disposed on the handle 4. In some embodiments, the elastic force prompt member 10 may include a display for displaying a value of the elastic force. In some embodiments, when the value of the elastic force exceeds a set threshold, the elastic force prompt member 10 may issue an alarm, such as displaying a warning image, making a warning sound, or the like to remind the user to control the operation strength. The set threshold may be a fixed value or may be determined according to different types of nerve to be detected. For example, a threshold for a cranial nerve may be set relatively low (e.g., 0.8N) since the cranial nerve is relatively sensitive; a threshold for a laryngeal nerve may be set as 1.2N; and a threshold for a nerve in the face, hands, feet, or knees may be set as 3 N.

In some embodiments, the nerve detecting device of the present disclosure may be coupled to a nerve monitor (not shown). In some embodiments, one end of a wire 5 may be connected to the probe 7, and the other end may be connected to the nerve monitor through a socket 6. In some embodiments, the elastic force prompt member 10 may be disposed in the nerve monitor. Specifically, the nerve monitor may receive an electrical signal about the value of the elastic force of the elastic member generated by the elastic force measuring member 11, and generate prompt information about the value of the elastic force of the elastic member. For example, the nerve monitor may include a display, which may display the value of the elastic force. In addition to the text display, the nerve monitor may prompt the value of the elastic force in the form of images, speech, or the like. Since the elastic force prompt member is used, a user (such as a doctor) can conveniently know that the pressure applied to a patient when using the nerve detecting device of the present disclosure. Thus, the use of strength may be controlled to ensure the reliable contact between the probe and a nerve or tissue, and at the same time protect a patient's nerve or tissue from damage.

In some embodiments, different types of the nerve detecting device may have different maximum values of the elastic force. For example, elastic members having different elastic coefficients may be used to illustrate differentiation of maximum values of the elastic force. Specifically, according to Hooke's law:

F=k*X (1)

wherein, F is the value of the elastic force of the elastic member, k is the elastic coefficient of the elastic member, and X is the value of the elastic deformation of the elastic member. As can be seen from Formula (1), when the same elastic deformation X occurs, the generated elastic forces are different for elastic members with different elastic coefficients k. Accordingly, in the case of a fixed maximum elastic deformation, different maximum values of the elastic force may be achieved by selecting elastic members with different elastic coefficients. In some embodiments, nerve detecting devices having different maximum values of the elastic force may be selected for different types of surgery. For example, for a high sensitive nerve, a nerve detecting device having a relatively small maximum value of the elastic force may be selected; and for a low sensitive nerve, a nerve detecting device having a relatively high maximum value of the elastic force may be selected. Merely by way of illustration, a nerve detecting device having a maximum value of the elastic force of 0.8N may be selected for a cranial nerve; a nerve detecting device having a maximum value of the elastic force of 1.2N may be selected for a laryngeal nerve; and a nerve detecting device having a maximum value of the elastic force of 3N may be selected for a nerve in the face, hands, feet, or knees. In some embodiments, nerve detecting devices having different value of the elastic force s may be selected for different individuals. For example, a nerve detecting device having a relatively small value of the elastic force may be selected for a patient with high sensitivity; and a nerve detecting device having a relatively high value of the maximum elastic force may be selected for a patient with low sensitivity.

The elastic force measuring member 11 may convert the value of the elastic force of the elastic member 8 to an electrical signal. In some embodiments, the elastic force measuring member 11 may include an adjustable resistor connected to the elastic member 8. The change in the length of the elastic member 8 may change the resistance of the adjustable resistor, thereby achieving converting the value of the elastic force to the electrical signal. For example, the value of the elastic force may be positively correlated with the resistance value, or the value of the elastic force may be negatively correlated with the resistance value. In some embodiments, the elastic force measuring member 11 may include a pressure sensor, and the pressure sensor may measure the value of the elastic force of the elastic member 8. Specifically, when the nerve detecting device is in use, and the probe head 1 is in contact with the human body and is subjected to pressure from the human body, the elastic member 8 may be compressed to exert the pressure to the pressure sensor. According to the pressure value measured by the pressure sensor, the value of the elastic force of the elastic element 8 may be obtained.

In some embodiments, the elastic member 8 may be also connected to an elastic force adjustment member (not shown). The elastic force adjustment member may be used to adjust the maximum value of the elastic force of the elastic member 8. For example, the maximum value of the elastic force may be adjusted to change the elastic force by limiting a scalable length of the elastic member 8. The maximum value of the elastic force of the elastic element 8 may be adjusted, by the elastic force adjustment member, to match maximum values of the elastic force of different types of surgery. For example, for a cranial nerve, the maximum value of the elastic force of the elastic member 8 may be adjusted as 0.8N; for a laryngeal nerve, the maximum value of the elastic force may be adjusted as 1.2N; and for a nerve of the face, hands, feet, or knees, the maximum value of the elastic force may be adjusted as 3N.

In some embodiments, the elastic member 8 may be made of a conductive material. The conductive material may include a metal, conductive rubber, conductive non-metal, conductive alloys, or the like, or any combination thereof. In some embodiments, the maximum value of the elastic force of the elastic member 8 may be adjusted for different individuals. For example, for a patient with high sensitivity, the maximum value of the elastic force may be adjusted down; and for a patient with low sensitivity, the maximum value of the elastic force may be adjusted up.

In some embodiments, a current adjustment member 9 may be provided on the handle 4. The current adjustment member 9 may be used to regulate a value of a nerve stimulation current. In some embodiments, the current adjustment member 9 may be electrically connected to the nerve monitor through a wire. The nerve monitor may receive a current adjustment signal sent by the current adjustment member 9 and then control the value of the output stimulation current. For example, the nerve monitor may include a host and a current output part. The host may be used to receive the current adjustment signal sent by the current adjustment member 9, generate a current control signal according to the current adjustment signal and send the current control signal to the current output part. A current output unit may output a corresponding current according to the received current control signal. In some embodiments, the current output unit may include a voltage/current conversion integrated circuit that converts an input voltage into a current output. Specifically, after the host of the monitor receives a current adjustment signal, a microcontroller unit (MCU) of the host may control a value of the input voltage in the voltage/current conversion integrated circuit by controlling the pulse width modulation (PWM) wave. Through the voltage/current conversion of the integrated circuit, an appropriate current may be outputted.

In some embodiments, different stimulation currents can be adjusted for different types of nerves. For example, for a cranial nerve, a stimulation current may be adjusted to 0 mA-0.5 mA; for a laryngeal nerve, a stimulation current may be adjusted to 0.5 mA-10 mA; and for a nerve of the face, hands, feet, or knees, a stimulation current may be adjusted to 10 mA-30 mA. In some embodiments, due to the difference in sensitivity of different individuals, stimulation currents may be adjusted. For example, for a patient with high sensitivity, a stimulation current may be adjusted down; and for a patient with low sensitivity, a stimulation current may be adjusted up.

In some embodiments, a maximum current threshold may be set. The limiting stimulation current may be limited to less than or equal to the maximum current threshold to ensure the safety of the nerve or tissue. For example, the maximum current threshold may be 40 mA, 35 mA, 30 mA, 25 mA, 20 mA, or the like. In some embodiments, different maximum current thresholds may be set for different types of nerves. For example, for a cranial nerve, a maximum current threshold may be adjusted to 0.5 mA; for a laryngeal nerve, a maximum current threshold may be adjusted to 10 mA; and for a nerve of the face, hands, feet, or knees, a maximum current threshold may be adjusted to 30 mA. In some embodiments, different maximum current thresholds may be set for different individuals. For example, for a patient with high sensitivity, a maximum current threshold may be set relatively low; for a patient with lower sensitivity, the maximum current threshold may be set relatively high.

The current adjustment member 9 may be in various forms including but not limited to, a button, a knob, a touch key, or the like. In some embodiments, as shown in FIGS. 13 and 14, the current adjustment member 9 may be two buttons for adjusting up and down the current, respectively. The adjustment step may be a fixed value or an unfixed value. In some embodiments, different adjustment steps may be set for different stimulation current ranges. It can be understood that a relatively small stimulation current requires higher adjustment accuracy, and a relatively small adjustment step is set to achieve high-precision adjustment. For example, in the range of 0 to 0.5 mA, the adjustment step may be 0.01 mA; in the range of 0.5 mA to 1 mA, the adjustment step may be 0.1 mA; in the range of 1 mA to 10 mA, the adjustment step may be 0.5 mA; and in the range of 10 mA to 30 mA, the adjustment step may be 1 mA. It should be noted that the two buttons shown in FIGS. 13 and 14, respectively, are only examples of the current adjustment member, and are not intended to limit the present disclosure. In some embodiments, other forms of the current adjustment member may be set. For example, four buttons may be set, two of which are used to roughly adjust the stimulation current with a first step length (to increase or decrease), and the other of which are used to precisely adjust the stimulation current with a second step length. The second step length may be smaller than the first step length.

In some embodiments, the nerve detecting device of the present disclosure may further include a stimulation current prompt member for prompting the value of the stimulation current. The value of the stimulation current may be prompted in various forms, including but not limited to, text, images, voice, or the like. In some embodiments, the stimulation current prompt member may be disposed on the handle 4. For example, a display may be provided on the handle 4 to display the value of the stimulation current. In some embodiments, the stimulation current prompt member and the aforementioned elastic force prompt member may be integrated into the same component, or may be individual components. In some embodiments, the stimulation current prompt member may also be disposed on the nerve monitor. For example, the display of the nerve monitor may display the value of the stimulation current.

In some embodiments, the probe 7 may also include a sleeve 2.

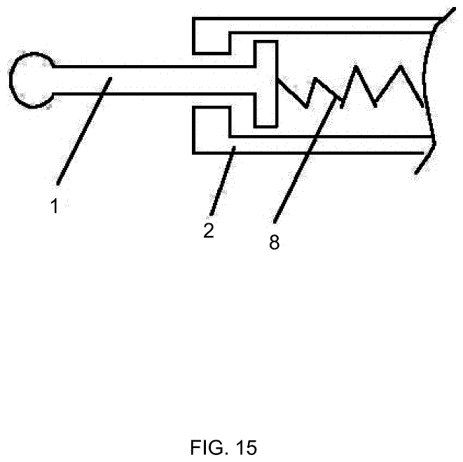

FIG. 15 is a schematic diagram of a connection structure between a probe head 1 and a sleeve 2 according to some embodiments of the present disclosure.

As shown in FIGS. 13 and 15, the elastic member 8 may be mounted within the sleeve 2, and one end of the probe 7 may be inserted into a first end of the sleeve 2 to connect with the elastic member 8. A second end of the sleeve 2 may be connected to the handle 4. In some embodiments, the sleeve 2 may be made of a conductive material, and the wire 5 may be electrically connected to the sleeve 2, thereby achieving electrical connection of the wire 5 and the probe 7. In some embodiments, a surface of the sleeve 2 may be provided with an insulating layer 3, and the insulating layer may be a heat shrinkable sleeve or an insulating coating. In some embodiments, the probe head 1 may be in a spherical head shape. In some embodiments, in order to prevent the probe head 1 from sliding from the sleeve 2, in addition to make the probe head 1 in a welding connection with the elastic member 8, an anti-skid step may be provided on one end of the inserting sleeve 2, and a limit step matched with the anti-skid step may be provided on an inner wall of the sleeve 2. When installing, the probe head 1 may be inserted into the sleeve 2 from the other end of the sleeve 2, after the step on the probe collides with the step inside the sleeve, a head of the probe head 1 may be subjected to spherical upsetting. In addition, after an end of the probe with the step is inserted into the sleeve 2, an end of the sleeve may be turned inward to form a stop inner step.

In some embodiments, the nerve detecting device of the present disclosure may further include a probe monitoring member (not shown) for monitoring the use of probe 7 and generating probe monitoring information. For example, the probe monitoring member may monitor a cumulative usage time of the probe. Merely by way of example, the probe monitoring member may read the cumulative usage time of the probe from electrically erasable programmable read only memory (EEPROM) or write the cumulative usage time of the probe into EEPROM. As another example, the probe monitoring member may monitor the elasticity of the elastic member in the probe. In some embodiments, the probe monitoring member may give a prompt in response to the determination that the probe monitoring information satisfies a set condition. For example, when the cumulative usage time exceeds a certain duration, or the elasticity of the elastic member is attenuated to a certain extent, the probe monitoring member may issue an alert to prompt the user to replace the elastic member in time. In some embodiments, the probe monitoring member may be provided on the handle 4. In other embodiments, the probe monitoring member may be integrated into the nerve monitor.

The benefits of the present disclosure may include, but is not limited to the following aspects. (1) Since the nerve monitoring device without the nerve stimulation module integrated therein may be designed as an integrated device in a relatively small volume, not only does it cause little hindrance to the surgical operation, but also facilitates the surgeon to operate the nerve monitoring device closely. (2) By disposing the output module on the host 210, there may be little effect on the overall volume of the nerve monitoring device, which greatly improves the convenience of using the nerve monitoring device. (3) The electrode can be directly connected to the host of the nerve monitoring device, avoiding the noise and space occupancy problems caused by the electrode transmission line. It should be noted that the beneficial effects of different embodiments may be different. In various embodiments, the beneficial effects may include any combination of one or more of the above or any other possible advantage effect.

The above embodiments may be combined to each other to obtain more embodiments. For example, the nerve monitoring device shown in FIGS. 2 and 3 may be used together with the nerve detecting device shown in FIGS. 13 and 14 to position a nerve. In other words, the nerve monitoring device shown in FIGS. 2 and 3 and the nerve detecting device shown in FIGS. 13 and 14 may be combined into a nerve monitoring system, and details are not described herein again.

The basic concepts have been described above, apparent to those skilled in the art, and the above disclosure is not construed as limiting the present disclosure. Although not explicitly stated here, those skilled in the art may make various modifications, improvements and amendments to the present disclosure. These alterations, improvements, and modifications are intended to be suggested by this disclosure, and are within the spirit and scope of the exemplary embodiments of this disclosure.

Moreover, certain terminology has been used to describe embodiments of the present disclosure. As “one embodiment”, “one embodiment”, and/or “some embodiments” means a certain feature, structure or feature of the present disclosure at least one embodiment. Therefore, it should be emphasized and noted that “one embodiment” or “one embodiment” or “an alternative embodiment” or “an alternative embodiment” mentioned in this specification is not necessarily referred to as the same embodiment. In addition, some features, structures, or features in the present disclosure of one or more embodiments may be appropriately combined.

Moreover, those skilled in the art can understand that various aspects of this application can be illustrated and described through a number of patentable categories or situations, including any new and useful process, machine, product or combination of substances, or any new and useful improvement to them. Accordingly, all aspects of the present disclosure may be performed entirely by hardware, may be performed entirely by softwares (including firmware, resident softwares, microcode, etc.), or may be performed by a combination of hardware and softwares. The above hardware or softwares can be referred to as “data block”, “module”, “engine”, “unit”, “component”, or “system”.

Moreover, unless otherwise stated in the claims, the order of the processing elements and sequences of the present disclosure, the use of digital letters, or other names are not intended to limit the order of the application processes and methods. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, this disclosure does not mean that the present disclosure object requires more features than the features mentioned in the claims. Rather, claimed subject matter may lie in less than all features of a single foregoing disclosed embodiment.

In some embodiments, a number of descriptive components, attributes, should be understood, such for the numbers described in the embodiments, in some examples, used modified words “approximately”, “approximation” or “generally” Modified. Unless otherwise stated, “approximately”, “approximate” or “substantially” indicates that the number is allowed to have a change of ±20%. Accordingly, in some embodiments, the numerical parameters used in the specification and claims are approximately values, and the approximation can change according to the characteristics required by the individual embodiments. In some embodiments, the numerical parameters should consider the predetermined effective digits and use the general bits reserved. Although the numerical domains and parameters used in the present disclosure are used to confirm the wide range of ranges, the settings of such values are as accurately as possible within the feasible range in the specific embodiments.

For each patent, patent application, patent application publications and other materials referenced by the present disclosure, such as articles, books, instructions, publications, documents, etc., hereby incorporated herein by reference. Except for the application history documents of inconsistent or conflicting the content of the present disclosure, there is also a limited file (currently or after the present disclosure) of the present disclosure (currently or later). It should be noted that if a description, definition, and/or terms in the apparatus of the present disclosure are inconsistent or conflict with the contents of the present disclosure, the use of the present disclosure, definition, and/or the term.

At last, it should be understood that the embodiments described in the present disclosure are merely illustrative of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

1. A monitoring device, comprising:
a host;
an information acquisition module connected to the host via an electrical signal, the information acquisition module being configured to acquire an electromyographic signal from a target area, the host including a signal processing module, the signal processing module being configured to process the electromyographic signal to determine monitoring information corresponding to the electromyographic signal; and
an output module connected to the signal processing module via an electrical signal, the output module at least being configured to output the monitoring information.

2. The monitoring device of claim 1, wherein
the information acquisition module includes an electrode, the electrode being configured to acquire the electromyographic signal generated by an external stimulation and transmit the electromyographic signal to the signal processing module.

3. The monitoring device of claim 2, wherein a value of the electromyographic signal acquired by the electrode ranges from 5 μV to 1 mV.

4. The monitoring device of claim 2, wherein the electrode transmits the electromyographic signal to the signal processing module in a wire or wireless manner.

5. The monitoring device of claim 2, wherein a material of the electrode includes medical stainless steel and high conductive rubber.

6. The monitoring device of claim 2, wherein a connection interface is provided on the host, the connection interface being configured to make the electrode in a direct connection with a main board of the host.

7. The monitoring device of claim 6, wherein the direct connection includes a pluggable connection.

8. The monitoring device of claim 7, wherein the connection interface is further configured to make one end of an electrode transmission line in a pluggable connection with the host, the other end of the electrode transmission line being connected with one end of the electrode via an electrical signal, the other end of the electrode being near to the target area.

9. The monitoring device claim 1, wherein the output module includes an alarm unit, and in response to determining that the monitoring information exceeds a preset threshold, the alarm unit performs an alarm prompt.

10. The monitoring device of claim 1, wherein the output module is disposed on the host.

11. The monitoring device of claim 10, wherein the host is fixable on a surgical bed.

12. The monitoring device of claim 2, wherein the electrode includes a pin type electrode of which a length ranges from 4 cm to 10 cm.

13. The monitoring device of claim 1, wherein the host further includes a threshold adjustment unit connected to the signal processing module via an electrical signal, the threshold adjustment unit being configured to pre-adjust a threshold associated with the electromyographic signal.

14. The monitoring device of claim 1, wherein a maximum size of the host of the monitoring device is less than 50 mm.

15. A method for controlling a threshold of the monitoring device of claim 1, comprising:
setting a threshold of the monitoring device associated with the electromyographic signal;
placing an electrode of the information acquisition module to the target area, the electrode being configured to acquire the electromyographic signal and transmit the electromyographic signal to the signal processing module; and
starting the monitoring device to output, based on the threshold, the monitoring information corresponding to the electromyographic signal.

16. The method of claim 15, wherein
the monitoring device includes a first adjustment member and a second adjustment member for setting the threshold.

17. The method of claim 15, wherein setting the threshold of the monitoring device includes:
setting the threshold by a text input box of the monitoring device.

18. The method of claim 16, wherein setting the threshold of the monitoring device includes:
triggering the first adjustment member to increase the set threshold; or
triggering the second adjustment member to decrease the set threshold.

19. The monitoring device of claim 1, wherein the monitoring information corresponding to the electromyographic signal indicates whether the electromyographic signal satisfies a preset condition and/or a parameter value of the electromyographic signal.

20. The monitoring device of claim 19, wherein the preset condition includes that a voltage peak of the electromyographic signal exceeds a set voltage threshold.
